(19) United States
(12) Patent Application Publication
(10) Pub. No.: US 2006/0133997 A1
(43) Pub. Date: Jun. 22, 2006

(54) A method of demonstrating draining activity of a cosmetic and/or dermocosmetic treatment on the superficial dermis and/or the epidermis

(75) Inventors: Bernard Querleux, Perreux (FR); Roland Bazin, Bievres (FR)

Correspondence Address: OLIFF & BERRIDGE, PLC, P.O. BOX 19928, ALEXANDRIA, VA, 22320, US

(73) Assignee: L'OREAL, Paris (FR)

(21) Appl. No.: 10/551,132

(22) PCT Filed: Feb. 2, 2004
(86) PCT No.: PCT/FR04/00232

§ 371 Date: Dec. 13, 2005

(30) Foreign Application Priority Data
Apr. 9, 2003 (FR) 03/04399

(51) Int. Cl. A61K 49/00; G06F 19/00; A61K 8/02

(52) U.S. Cl. 424/9.1; 424/401; 702/19

(57) ABSTRACT

The present invention concerns a method of demonstrating the draining activity of a cosmetic and/or dermocosmetic treatment, said method comprising: prior to the treatment, acquiring at least one first datum representative of the water content in the superficial dermis and/or in the epidermis, using an MRI imaging technique having high resolution; treating at least part of the body with the composition; after the treatment, acquiring at least one second datum representative of the water content in the superficial dermis and/or epidermis, using said MRI imaging technique; demonstrating any draining activity by comparing the first and second data.

The present invention relates to the field of cosmetics and/or dermocosmetics and in particular to compositions having a draining action, non-exclusive examples of which are certain slimming products or anti-cellulite products. The invention is also important to the study of the effects of such cosmetic and/or dermocosmetic compositions. The term “cosmetic composition” as used herein means a composition as defined in Directive 93/35/EEC dated 14^{th} June 1993, amending Directive 76/768/EEC. The term “cosmetic composition” is used herein to designate both cosmetic compositions and/or dermocosmetic compositions.

### BACKGROUND OF THE INVENTION

Many cosmetic compositions claiming a slimming action are known; they act by limiting lipogenesis or encouraging lipolysis.

It can prove fairly difficult to demonstrate the effects induced by applying a composition claiming a draining action, in particular a slimming action; in particular it is difficult to quantify such effects.

It can also prove difficult to demonstrate quantifiable effects over a relatively short time period.

### OBJECTS AND SUMMARY OF THE INVENTION

In a first aspect, the invention aims to provide a method of demonstrating the draining activity of a cosmetic treatment comprising the use of a cosmetic composition.

Within the context of the present invention, the term “draining activity” means the action of reducing water retention in the epidermis and/or superficial dermis.

Thus, in a first aspect, the invention provides a method of demonstrating the draining activity of a treatment, said method comprising:

prior to the treatment, acquiring at least one first datum representative of the water content in the superficial dermis and/or in the epidermis, using a magnetic resonance imaging (MRI) technique having high spatial resolution;

treating at least part of the body with the composition, in particular by topical or general means;

after the treatment, acquiring at least one second datum representative of the water content in the superficial dermis and/or in the epidermis, using said MRI imaging technique;

demonstrating any draining activity by comparing the first and second data.

The term “by general means” preferably means by ingestion or inhalation.

The term “MRI imaging technique having high spatial resolution” means an MRI technique, in particular proton imaging, with spatial resolution that is sufficient to distinguish the epidermis from the superficial dermis on the MRI image obtained. Typically, a sufficient depth spatial resolution is of the order of 50 μm (micrometers) or better, preferably 35 μm or better.

The term “superficial dermis” means the portion of the dermis that extends between the epidermis and the deep dermis into which adipose tissue indentations may extend. In some individuals, the superficial dermis may extend to a depth in the range 50 μm to 500 μm from the surface of the skin.

The above treatment may comprise topical application of the composition, possibly comprising at least one active ingredient having an action on water retention in the superficial layers of the skin. The term “superficial layers of the skin” designates the superficial dermis and the epidermis.

Alternatively, the treatment may be carried out using an oral cosmetic or by inhalation.

If appropriate, the treatment may advantageously further comprise a massage, in particular a massage which can act on lymphatic circulation.

To demonstrate the draining activity, it may be advantageous to compare N(H) before and after treatment. N(H) designates the relative proton density and corresponds to the fraction of protons detectable by magnetic resonance imaging.

The parameter N(H) is well known to the person skilled in the MRI art and is defined in the article “Characterization of the skin in vivo by high resolution magnetic resonance imaging: water behaviour and age-related effects”, S. Richard et al, The Journal of Investigative Dermatology, vol 100, No 5, May 1993, the contents of which are hereby incorporated by reference.

However, other parameters are representative of water content. These are in particular the parameters T1 and T2 which describe interactions between protons, in particular those in water, and their environment. More particularly, an increase in water content is often associated with an increase in T1, as reported in the following article, for example: “In vivo brain water determination by T1 measurements: effect of total water content, hydration fraction, and field strength”, P. P. Fatouros et al, Magnetic Resonance in Medicine 17: 402-413, 1991.

Said parameters T1 and/or T2 may be used independently of N(H) or as a complement to N(H).

When the treatment comprises topical cutaneous application of a composition, said composition may include a lipolytic active ingredient, in particular one of those identified below.

The above method may provide a demonstration of an unexpected draining activity linked to the use of certain active ingredients, the action of which on the water content of the superficial skin layers may not a priori be anticipated.

In a further aspect, independently or in combination with the above, the invention also provides a composition containing, in a cosmetically acceptable medium, at least one active ingredient, in particular a lipolytic active ingredient, said active ingredient being such that when it is present in sufficient quantities in the composition, it may cause a reduction in N(H) of at least 2.5% in the superficial dermis and/or the epidermis and/or at least 2% for T1, and/or at least 1.5% for T2 in the epidermis. The reduction in N(H) may in particular be at least 4% in the superficial dermis and/or the epidermis, preferably at least 9% in the superficial dermis. The optimum concentration may be determined experimentally, in particular by successive measurements of T1 and/or T2 and/or N(H)

The draining activity may essentially be due to a single active ingredient or, in a variation, to a plurality of active ingredients the individual concentrations of which may be lower.

The term “cosmetically acceptable” designates a composition that is compatible at least with the skin.

Advantageously, the draining power of the composition is sufficiently strong for the reduction in N(H), and/or T1, and/or T2 as defined above, to be observed over a relatively short period of treatment, for example four weeks, with daily applications morning or evening, for example.

The composition may include at least one active ingredient selected from caffeine and its derivatives, caffeine citrate, theophylline and its derivatives, theobromine, acefylline, aminophylline, chloroethyltheophylline, diprofylline, diniprophylline, etamiphylline and its derivatives, etofylline, proxyphylline, ephedrine and its derivatives, combinations of caffeine and silanol, compounds of natural origin containing xanthic bases such as extracts of tea, coffee, guarana, maté, kola (Cola Nitida); plant extracts of Garcinia Cambogia, extracts of Bupleurum chinensis, extracts of common ivy (Hedera Helix), arnica (Arnica Montana L), rosemary (Rosmarinus officinalis L), marigold (Calendula officinalis), sage (Salvia officinalis L), ginseng (Panax ginseng), St John's Wort (Hypericum Perforatum), Butcher's Broom (Ruscus aculeatus L), meadowsweet (Filipendula ulmaria L), orthosiphon (Orthosiphon Stamincus Benth), birch (Betula alba), extracts of pumpwood and argan tree, extracts of ginkgo biloba, extracts of horsetail, extracts of escin, complexes of phospholipids and of proanthocyanidines from horse chestnut bark, extracts of cangzhu, extracts of chrysanthemum indicium, sapogenins such as diosgenin or hecogenin, their derivatives and natural extracts containing them, in particular Wild Yam, extracts from plants of the genus Armeniacea, Atractylodis Platicodon, Sinom-menum, Pharbitidis, Flemingia, extracts of Coleus such as C Forskohlii, C blumei, C esquirolii, C scutellaroides, C xanthantus and C Barbatus, extracts of Ballota, extracts of Guioa, Davallia, Terminalia, Barringtonia, Trema, Antirobia, extracts from algae or phytoplankton such as rhodysterol or extract of Laminaria Digitata, the alga skeletonema, and diatoms.

The composition may in particular include at least one extract of Dioscorea which is rich in diosgenin, deriving, for example, from wild yam tubers. This extract or any other active ingredient having a draining activity may, for example, be present in the composition along with at least one glyceride of a fatty acid or mixture of C₆ to C₁₂ fatty acids, optionally polyoxyethylenated and/or polyoxypropylenated.

As an example, it may be possible to select an extract of Dioscorea opposita tubers in solution in a mixture of a derivative of polyethylene glycol (60E) and caprylic and capric acids mono-, di- and tri-glycerides/preservatives/glycerin (weight ratio 1/93.8/0.2/5), sold by SEDERMA under the trade name “Dioschol”, in particular in a concentration of 5% or more, preferably 8%, with respect to the total composition weight.

The invention also provides the use of a lipolytic active ingredient, for example an extract of tubers of Dioscorea opposita such as that described above, for the production of a composition having a draining effect on the superficial dermis and/or the epidermis. This draining effect may be claimed as such.

In a further aspect, the invention pertains to a method of promoting the sale of a cosmetic composition, which highlights the draining activity, in particular in the superficial dermis and/or the epidermis, demonstrated by a magnetic resonance imaging technique.

Any communications channel could be used for the promotion. It may in particular be carried out by a retailer, directly at the point of sale, over the radio, on the television or by telephone, in particular in the context of commercials or short messages. It may also be carried out through the press or by means of any other document, in particular for advertising purposes. It may also be carried out via the Internet, via any other suitable information network or via a mobile telephone network. It may also be placed directly on the packaging or on any other explanatory note associated with the composition.

### BRIEF DESCRIPTION OF THE DRAWINGS

The invention can be better understood from the following detailed description made with reference to the accompanying drawings in which:

FIG. 1 diagrammatically shows the acquisition of an MRI image for an individual;

FIG. 2 shows an example of a MRI image;

FIG. 3 shows an example of the change in MRI signal in a region of interest in a cutaneous layer as a function of repetition time Tᵣ of the sequence, enabling calculation of T1 by exponential approximation;

FIG. 4 shows an example of the change in MRI signal in a region of interest within a cutaneous layer as a function of the echo time Tₑ of the sequence, enabling calculation of T2 by exponential approximation; and

FIGS. 5 to 7 illustrate an example of the technique for applying the composition.

### MORE DETAILED DESCRIPTION

To demonstrate the draining activity of a cosmetic composition applied to the skin, it is possible to acquire MRI images using the following protocol.

Measuring Protocol

By way of example, the proton magnetic resonance imaging apparatus used is the SIGNA 1.5 Tesla apparatus from General Electric.

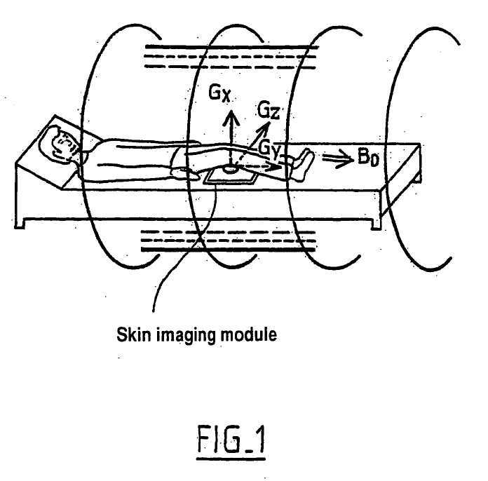

The test subject lies in the apparatus as shown in FIG. 1. In the figure, Gₓ, G_{y}, and G_{z} conventionally designate the intensity gradients in the three respective directions.

If the apparatus does not initially have sufficient spatial resolution as regards depth, for example 35 μm or better, it may be equipped with a skin imaging module such as that described in French patent application FR-A-2 612 641 the contents of which are hereby incorporated by reference, said module being intended to improve the spatial resolution of MRI images. One example of the use of such a module is described in the article “In vivo proton relaxation times analysis of the skin layers by Magnetic Resonance Imaging”, S. Richard et al, The Journal of Investigative Dermatology, vol. 97, No. 1, July 1991, 120-125, the contents of which are hereby incorporated by reference.

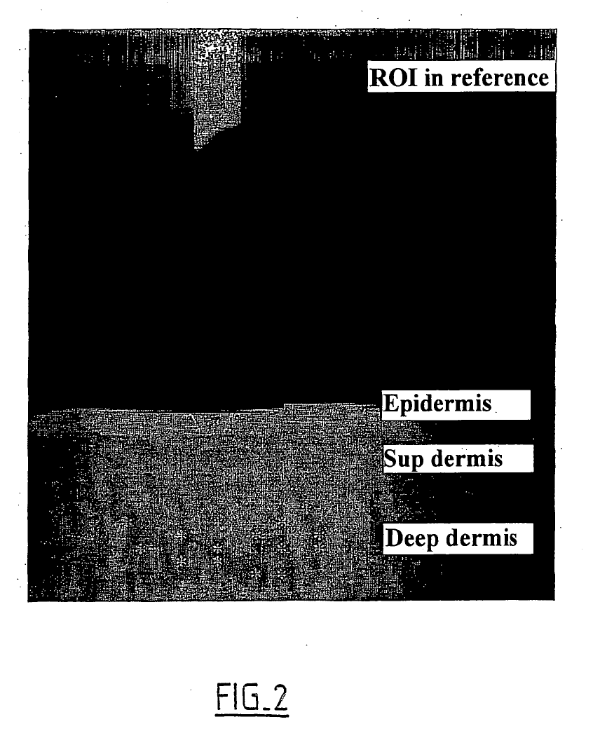

A small tube of non magnetic material filled with demineralized water is placed close to the study region so that it appears on the MRI image and acts as a reference, as can be seen in FIG. 2.

For each image, various measurements can be made in different regions of interest. The term “region of interest, abbreviated to ROI” means a zone of the image in which a measurement of the mean signal intensity is made.

Reference

The region of interest is defined by a simple rectangle, as can be seen in FIG. 2.

Epidermis

The region of interest is defined by three rectangles disposed substantially end to end, as illustrated in FIG. 2, through the thickness of the epidermis.

Superficial Dermis

The region of interest is defined by three rectangles disposed substantially end to end between the epidermis and the deep dermis, into which the indentations of the adipose tissue extend.

Calculation of Relaxation times T1 and T2

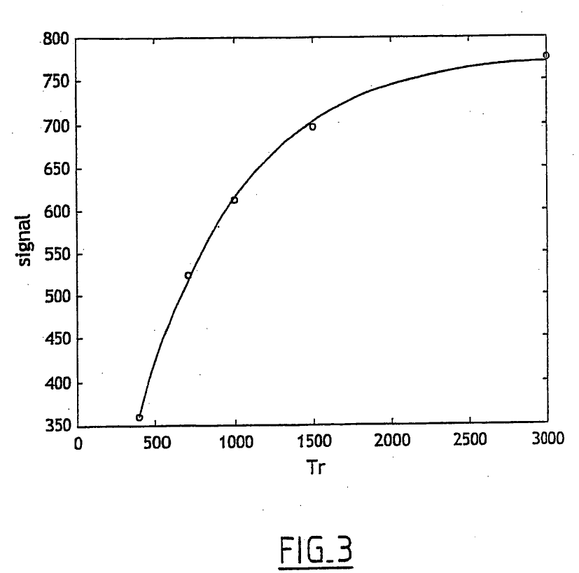

For each region of interest, five images are acquired by varying the repetition time Tᵣ, with Tᵣ being respectively 3000 ms, 1500 ms, 1000 ms, 700 ms, and 400 ms (milliseconds). An example of the curve obtained is shown in FIG. 3. The value of the relaxation time T1 that best describes the exponential variation observed is calculated in a manner that is known per se.

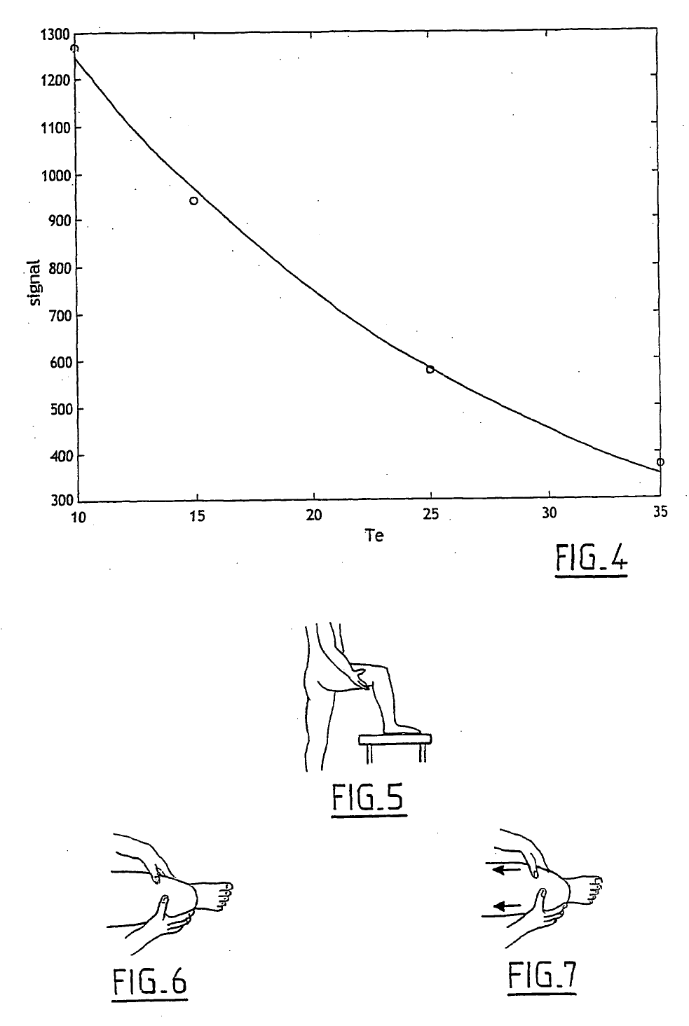

Four images are also acquired to measure the relaxation time T2, by varying the echo time Tₑ, this being, for example, equal to 10 ms, 15 ms, 25 ms, and 35 ms in succession. An example of the curve obtained is shown in FIG. 4. The value of the relaxation time T2 that best describes the observed exponential variation is calculated in a manner that is known per se.

Subsequently, for each region of interest (epidermis or superficial dermis), the proton density Rho and relative density N(H) are calculated from the following formulae:

$$Rho = \frac{{S}_{( Te = 10 ms , Tr = 3000 ms )}}{Exp ( - 10 / T 2 ) * ( 1 - exp ( - 3000 / T 1 ) )}$$

N(H)=Rho_{region of interest}/Rho_{reference}

In the above formulae, S designates the mean signal intensity in the region of interest in question for the acquired image with Tₑ=10 ms and Tᵣ=3000 ms.

The proton density Rho may be considered to be representative of the water content, but it also depends on a factor linked to the acquisition conditions, while N(H) only depends on the water content in the study tissue. Normalization makes it possible to compare between individuals or any one individual at different times.

Thus, a reduction in N(H) can reveal a reduction in water content in the superficial layers of the skin, and thus a draining effect.

Similarly, a reduction in T1 or T2 can reveal a reduction in the water content in the superficial layers of the skin, and thus a draining effect.

Tests

T1 and T2 were measured and N(H) was calculated using the protocol described above for the superficial dermis and epidermis of 20 female volunteers aged 19 to 45 years, presenting with aesthetically displeasing localized excess fat such as cellulite in the thighs, visible to the naked eye, and a QUETELET (body mass) index in the range 20 to 27. The QUETELET index is the ratio W/H², where W is the weight in kg and H the height in meters (m).

The composition under evaluation was applied daily, morning or evening, over one month, to the hips and the legs, from the top of the thigh down to the knees using a predetermined sequence of hand movements for application over about three minutes, as illustrated in FIGS. 5 to 7.

The individual started by lifting one foot, placing it on an object such as a chair or bath to take up a comfortable position as shown in FIG. 5.

The individual then placed her hands either side of her knee and placed her thumbs on the front of her thigh, as illustrated in FIG. 6.

The individual then moved her hands smoothly up her thigh in a rapid, firm movement, one hand being moved to the top of the buttock, as illustrated in FIG. 7.

Finally, the hands were moved alternately forwards and backwards over the thigh until the composition had penetrated completely.

The parameters T1, T2 and N(H) were initially acquired for the epidermis and the superficial dermis before any application, then after four weeks of treatment.

The formulation (weight % with respect to the total composition weight) for the composition applied during the tests was similar to that given below.

| Aqueous phase | |
|---|---|
| Water | Qs 100 |
| Caffeine | 3 |
| Plant extract | 0.2 |
| Salicylic acid | 0.72 |
| Mg sulfate | 0.7 |
| Trisodium citrate | 2 |
| Glycerin | 8 |
| Butylene glycol | 5 |
| Dioschol(1) | 3 |
| Thermal water | 5 |
| Ethanol | 20 |
| Preservatives | 0.5 |
| Colorants | 0.0001 |
| Neutralizing agent | 0.72 |
| Oily phase | |
| Cyclopentasiloxane | 9 |
| Isoparaffin | 2 |
| Cyclohexasiloxane | 5 |
| Fragrance | 0.3 |
| DC2-5225C(2) | 8 |

(1)Dioschol: extract of Dioscorea opposita tuber (wild yam) in a mixture of a derivative of polyethylene glycol (60E) and caprylic and capric acids mono-, di-, and triglycerides/preservatives/glycerin (weight ratio 1/93.8/0.2/5) sold by SEDERMA.
(2)DC2-5225C: mixture of oxyethylenated, oxypropylenated polydimethylsiloxane (18OE/18OP), cyclopentasiloxane and water (weight ratio 10/88/2) sold by DOW CORNING.

The aqueous and oily phases were prepared separately when cold, then the aqueous phase was dispersed in the oily phase with vigorous stirring.

The significance of the results was determined for T1 and N(H) using a paired Student test. For T2, the significance of the results was determined by mixed covariance analysis with time as the fixed factor (experimental factor); the measurement of T2 in the reference at the same time was the covariable and the control factor was the random factor.

During the tests, it was established that there was no significant variation in the values T1, T2 and N(H) in the reference between the beginning and end of the treatment.

Epidermis

| | Start | End | Start/end change |
|---|---|---|---|
| T1 (ms) | 735 ± 82 | 711 ± 85 | p = 0.05˜−3.3% |
| T2 (ms) | 22.1 ± 1.2 | 21.8 ± 1.1 | p = 0.004˜−1.5% |
| N(H) (a.u.)* | 0.66 ± 0.03 | 0.63 ± 0.03 | p = 0.003˜−4.5% |

*a.u. = arbitrary units

Superficial dermis

| | Start | End | Start/end change |
|---|---|---|---|
| T1 (ms) | 691 ± 44 | 677 ± 46 | p = 0.03˜−2.0% |
| T2 (ms) | 12.8 ± 0.8 | 12.7 ± 0.8 | Not significant˜−0.8% |
| N(H) (a.u.)* | 0.42 ± 0.05 | 0.38 ± 0.04 | P < 0.0001˜−9.5% |

p designates confidence level and is often fixed at 5%, or p < 0.05

A statistically significant reduction in the water content can be seen in the superficial layers of the skin. This reduction is representative of draining activity.

The above measurement protocol could quantify the draining activity of a composition in terms of a variation in N(H), T1 or T2. It can thus demonstrate an unexpected activity on the superficial layers of the skin of a cosmetic composition including a lipolytic active ingredient.

The invention is not limited to a composition including a particular active ingredient and it covers any cosmetic composition including at least one active ingredient, in particular a lipolytic active ingredient, having a draining activity that may result in a relatively large reduction in N(H) and/or T1 and/or T2.

The composition may include at least one active ingredient having either an action on phosphodiesterase, by inhibiting it, on receptors to be inhibited, such as β-2 blockers, NPY blockers (in particular those described in EP-A-0 838 217), or on the synthesis of LDL or VLDL receptors, or stimulating β receptors and G proteins, leading to adenylcyclase activation.

The composition may also comprise a peptide, in particular a peptide derived from the parathyroid hormone as described in FR-A-2 788 058, FR-A-2 781 231, or a peptide described in FR-A-2 786 693, or any other peptide having lipolytic properties.

The composition may also comprise a protamine and its derivatives, for example a protamine such as that described in FR-A-2 758 724.

Clearly, in addition to at least one active ingredient claiming a draining effect, present in a quantity of 0.001% to 20%, preferably 0.1% to 10% by weight with respect to the total composition weight, for example, the composition may comprise other compounds, in particular adjuvants which are normal in the cosmetics and/or dermatological field, such as preservatives, antioxidants, complexing agents, solvents, fragrances, fillers, UV screens, bactericides, odor absorbers, coloring materials and lipid vesicles, said list not being limiting.

The composition may be packaged in a package which may or may not be thermoplastic, such as a pot, flask, or tube, in a quantity which may be in the range 5 mL (milliliter) to 250 mL.

If appropriate, the composition may be packaged in a device that can exert a massaging action on the skin during application.

The composition may be packaged with instructions regarding the massage to be carried out on application, said instructions appearing, for example, on the packaging itself or on a distinct element, for example a leaflet or a printed support.

Throughout the description, including in the claims, the expression “comprising a” should be understood as being synonymous with “comprising at least one”, unless specified to the contrary.

1. A method of demonstrating the draining activity of a cosmetic and/or a dermocosmetic treatment, said method comprising:
prior to the treatment, acquiring at least one first datum representative of the water content in the superficial dermis and/or in the epidermis, using an MRI imaging technique having high spatial resolution;
treating at least part of the body with the composition;
after the treatment, acquiring at least one second datum representative of the water content in the superficial dermis and/or in the epidermis, using said MRI imaging technique;
demonstrating any draining activity by comparing the first and second data.

2. A method according to claim 1, wherein the treatment comprises topical application of the composition.

3. A method according to claim 1, wherein the treatment comprises administration of said composition by general means, in particular ingestion or inhalation.

4. A method according to claim 1, wherein N(H) is compared before and after treatment.

5. A method according to claim 1, wherein the relaxation time T1 is compared before and after the treatment.

6. A method according to claim 1, wherein the relaxation time T2 is compared before and after the treatment.

7. A method according to claim 1, wherein the composition comprises a lipolytic active ingredient.

8. A cosmetic composition, containing, in a cosmetically acceptable medium, at least one active ingredient, in particular a lipolytic active ingredient, said active ingredient being such that when it is present in a sufficient quantity in the composition, use of said active ingredient in particular by topical or general means can cause a reduction in N(H) of at least 2.5% in the superficial dermis and/or the epidermis.

9. A cosmetic composition, containing, in a cosmetically acceptable medium, at least one active ingredient, in particular a lipolytic active ingredient, said active ingredient being such that when it is present in a sufficient quantity in the composition, use of said active ingredient in particular by topical or general means can cause a reduction in T1 of at least 2.0% in the superficial dermis and/or the epidermis.

10. A cosmetic composition, containing, in a cosmetically acceptable medium, at least one active ingredient, in particular a lipolytic active ingredient, said active ingredient being such that when it is present in a sufficient quantity in the composition, use of said active ingredient in particular by topical or general means can cause a reduction in T2 of at least 1.5% in the epidermis.

11. A composition according to claim 8, wherein variation in N(H) is at least 4% in the superficial dermis and/or the epidermis.

12. A composition according to claim 8, wherein the variation in N(H) is at least 9% in the superficial dermis.

13. A composition according to claim 8, including at least one active ingredient selected from caffeine and its derivatives, caffeine citrate, theophylline and its derivatives, theobromine, acefylline, aminophylline, chloroethyltheophylline, diprofylline, diniprophylline, etamiphylline and its derivatives, etofylline, proxyphylline, ephedrine and its derivatives, combinations of caffeine and silanol, compounds of natural origin containing xanthic bases such as extracts of tea, coffee, guarana, maté, kola (Cola Nitida); plant extracts of Garcinia Cambogia, extracts of Bupleurum chinensis, extracts of common ivy (Hedera Helix), arnica (Arnica Montana L), rosemary (Rosmarinus officinalis L), marigold (Calendula officinalis), sage (Salvia officinalis L), ginseng (Panax ginseng), St John's Wort (Hypericum Perforatum), Butcher's Broom (Ruscus aculeatus L), meadowsweet (Filipendula ulmaria L), orthosiphon (Orthosiphon Stamincus Benth), birch (Betula alba), extracts of pumpwood and argan tree, extracts of ginkgo biloba, extracts of horsetail, extracts of escin, complexes of phospholipids and of proanthocyanidines from horse chestnut bark, extracts of cangzhu, extracts of chrysanthemum indicium, sapogenins such as diosgenin or hecogenin, their derivatives and natural extracts containing them, in particular Wild Yam, extracts from plants of the genus Armeniacea, Atractylodis Platicodon, Sinom-menum, Pharbitidis, Flemingia, extracts of Coleus such as C Forskohlii, C blumei, C esquirolii, C scutellaroides, C xanthantus and C Barbatus, extracts of Ballota, extracts of Guioa, Davallia, Terminalia, Barringtonia, Trema, Antirobia, extracts from algae or phytoplankton such as rhodysterol or extract of Laminaria Digitata, the alga skeletonema, and diatoms.

14. A composition according to claim 8, wherein the variation in the water content is observed after four weeks of treatment.

15. A composition according to claim 8, comprising at least one extract of Dioscorea which is rich in diosgenin.

16. A composition according to claim 15, wherein the extract of Dioscorea derives from wild yam tubers.

17. A composition according to claim 8, comprising at least one fatty acid glyceride or a mixture of C₆ to C₂₂ fatty acids, optionally polyoxyethylenated and/or polyoxypropylenated.

18. A composition according to claim 8, comprising “Dioschol” in a concentration of 5% by weight or more, preferably 8% by weight or more with respect to the total composition weight.

19. A composition according to claim 8, the composition being packaged in a thermoplastic receptacle.

20. A composition according to claim 8, the composition being packaged in a receptacle other than a thermoplastic receptacle.

21. A composition according to claim 8, the composition being packaged with instructions describing a particular sequence of hand movements with which to apply it.

22. The use of a lipolytic active ingredient for the production of a composition having a draining effect on the superficial dermis and/or the epidermis.

23. A use according to claim 22, wherein the lipolytic active ingredient is selected from caffeine and its derivatives, caffeine citrate, theophylline and its derivatives, theobromine, acefylline, aminophylline, chloroethyltheophylline, diprofylline, diniprophylline, etamiphylline and its derivatives, etofylline, proxyphylline, ephedrine and its derivatives, combinations of caffeine and silanol, compounds of natural origin containing xanthic bases such as extracts of tea, coffee, guarana, maté, kola (Cola Nitida); plant extracts of Garcinia Cambogia, extracts of Bupleurum chinensis, extracts of common ivy (Hedera Helix), arnica (Arnica Montana L), rosemary (Rosmarinus officinalis L), marigold (Calendula officinalis), sage (Salvia officinalis L), ginseng (Panax ginseng), St John's Wort (Hypericum Perforatum), Butcher's Broom (Ruscus aculeatus L), meadowsweet (Filipendula ulmaria L), orthosiphon (Orthosiphon Stamincus Benth), birch (Betula alba), extracts of pumpwood and argan tree, extracts of ginkgo biloba, extracts of horsetail, extracts of escin, complexes of phospholipids and of proanthocyanidines from horse chestnut bark, extracts of cangzhu, extracts of chrysanthemum indicium, sapogenins such as diosgenin or hecogenin, their derivatives and natural extracts containing them, in particular Wild Yam, extracts from plants of the genus Armeniacea, Atractylodis Platicodon, Sinom-menum, Pharbitidis, Flemingia, extracts of Coleus such as C Forskohlii, C blumei, C esquirolii, C scutellaroides, C xanthantus and C Barbatus, extracts of Ballota, extracts of Guioa, Davallia, Terminalia, Barringtonia, Trema, Antirobia, extracts from algae or phytoplankton such as rhodysterol or extract of Laminaria Digitata, the alga skeletonema, and diatoms.

24. A use according to claim 22, wherein the active ingredient is an extract of Dioscorea which is rich in diosgenin.

25. A use according to claim 24, wherein the extract of Dioscorea extract derives from wild yam tubers.

26. A use according to claim 22, wherein the composition comprises at least one fatty acid glyceride or a mixture of C₆ to C₂₂ fatty acids, optionally polyoxyethylenated and/or polyoxypropylenated.

27. A use according to claim 22, wherein the composition comprises “Dioschol” in a concentration of 5% by weight or more, preferably 8% by weight or more with respect to the total composition weight.
